(19) United States
(12) Patent Application Publication
(10) Pub. No.: US 2025/0316352 A1
(43) Pub. Date: Oct. 9, 2025

(54) PERIODIC BEHAVIOR REPORT GENERATION METHOD AND APPARATUS, STORAGE MEDIUM, AND ELECTRONIC DEVICE

(71) Applicant: EVYD RESEARCH PRIVATE LIMITED, Singapore (SG)

(72) Inventors: Hong CHEN, Singapore (SG); Shuo DU, Singapore (SG)

(73) Assignee: EVYD RESEARCH PRIVATE LIMITED, Singapore (SG)

(21) Appl. No.: 17/624,102

(22) PCT Filed: Dec. 21, 2021
(86) PCT No.: PCT/IB2021/062068

§ 371 Date: Dec. 30, 2021

(30) Foreign Application Priority Data
Dec. 17, 2021 (CN) 202111545121.0
Dec. 17, 2021 (CN) 202111545122.5

(51) Int. Cl. G16H 15/00; G16H 10/60; G16H 20/10; G16H 20/30; G16H 20/60; G16H 50/30

(52) U.S. Cl. CPC G16H 15/00; G16H 10/60; G16H 20/10; G16H 20/30; G16H 20/60; G16H 50/30

(57) ABSTRACT

A periodic behavior report generation method, apparatus, and system, a storage medium, and an electronic device. The method includes: obtaining behavior data and a health index parameter of a user in a predetermined period, the behavior data including at least one of an exercise behavior, a dietary behavior, or a drug administration behavior; generating, according to the behavior data and the health index parameter, a periodic behavior report in the predetermined period for the user by at least partially using a machine learning model, the periodic behavior report including at least an evaluation result in the predetermined period, a behavior suggestion of a next period, and/or an index target of a next period; updating, based on the periodic behavior report, a behavior label of the user, and obtaining a patient education content matching with the behavior label; and sending the periodic behavior report and the patient education content to the user.

### CROSS-REFERENCE TO RELATED APPLICATION

The present application is filed based on a Chinese Patent Application No. 202111545122.5 filed on Dec. 17, 2021 and a Chinese Patent Application No. 202111545121.0 filed on Dec. 17, 2021, and claims priority to the Chinese Patent Applications, which are incorporated herein by reference in its entirety.

### BACKGROUND

In recent years, with the continuous development of the economy, people's diet mode also produces a great change, obesity and patients with a variety of diseases are continuously increasing. Proper diet, exercise, and drug administration behaviors help prevent obesity, overweight, and non-communicable and chronic diseases, including diabetes, hypertension, heart disease, stroke, and cancer. Among all kinds of patients, due to the large number of diabetic patients, continuous human intervention is needed, rendering a serious decline in the quality of life of many diabetic patients, and due to different situations of each patient, the patient life is at a loss, resulting in a serious burden to them.

For prevention or control of diabetes, it is crucial to intervene in behaviors such as diet, exercise, and drug administration. Diabetes patients can control blood glucose indexes well by controlling diet, adhering to regular exercises, taking drugs and testing blood glucose on time, thereby lowering impacts on the patients. Unfortunately, although there are currently blood glucose monitors and other devices for the patients, they only collect and monitor the user's blood glucose, but do not provide any guidance for the user's related behaviors, that is, after the user has persisted the exercise for a period of time, whether the exercise has any influence on the user's blood glucose control; when the user has taken drugs, whether the drug administration for the period of time brings any help to the blood glucose control of the user and whether the drug administration is excessive or underused are unknown by the user. Similarly, whether the dietary behavior has a relevant impact on the blood glucose and the like are also unclear for the user. In particular, the user needs to perform statistics on the function of the previous behaviors on the blood glucose control, and urgently wants to know how to exercise, take drug, diet, etc. in the next stage, i.e., the control effect of the historical behavior of the user on the blood glucose, and whether the current behavior should continue, etc., are desired by the user, but at present, there is no related technologies for generating a periodic behavior report for the user for reference.

### SUMMARY

In view of this, embodiments of the present application provide a periodic behavior report generation method, apparatus, and system, a storage medium, and an electronic device to at least solve the technical problems above existing in the prior art.

According to a first aspect of the present application, a periodic behavior report generation method is provided and includes: obtaining behavior data and a health index parameter of a user in a predetermined period, the behavior data including at least one of an exercise behavior, a dietary behavior, or a drug administration behavior; generating, according to the behavior data and the health index parameter, a periodic behavior report in the predetermined period for the user by at least partially using a machine learning model, the periodic behavior report including at least an evaluation result in the predetermined period, a behavior suggestion of a next period, and/or an index target of a next period; updating, based on the periodic behavior report, a behavior label of the user, and obtaining a patient education content matching with the behavior label; and sending the periodic behavior report and the patient education content to the user.

Preferably, the generating a periodic behavior report in the predetermined period for the user includes: inputting the exercise behavior into a preset exercise behavior model, to obtain an evaluation result of the exercise behavior of the user, and generating an exercise behavior suggestion of the next period for the user based on the evaluation result; where the exercise behavior model includes a plurality of sub-models and an integration module, and the integration module is configured to determine the evaluation result according to outputs of the plurality of sub-models.

Preferably, the generating a periodic behavior report in the predetermined period for the user includes: obtaining a dietary content image of the user, inputting the dietary content image into a dietary behavior model, to obtain an evaluation result of the dietary behavior of the user, and generating a dietary behavior suggestion of the next period for the user based on the evaluation result, where the dietary behavior model is a convolutional neural network.

Preferably, the generating a periodic behavior report in the predetermined period for the user includes: comparing a blood glucose index and/or body composition index in the health index parameter with corresponding thresholds, to obtain an evaluation result of the blood glucose index and/or body composition; and invoking a corresponding behavior suggestion template for the user according to the evaluation result and by combining the behavior data, to generate an index behavior suggestion of the next period for the user.

Preferably, the obtaining a patient education content matching with the behavior label includes: generating a personal label of the user based on the behavior data and the health index parameter of the user using an entity recognition algorithm; and performing label matching in a patient education content label library according to the personal label and using a patient education content of a highest degree of matching corresponding to at least one patient education content label as a matching patient education content.

Preferably, the generating a periodic behavior report in the predetermined period for the user includes: obtaining a latest value of the health index parameter generated by executing, by the user, a behavior guidance solution corresponding to the predetermined period in the predetermined period; obtaining basic physical data of the user, a current value of the health index parameter, and an execution result of executing, by the user, the behavior guidance solution corresponding to the predetermined period in the predetermined period; performing prediction processing on the current value, the basic physical data, and the execution result using a model, to obtain a predicted change value of a health index parameter of the user in the next period; and determining, based on the latest value and the predicted change value, an index target of the health index parameter of the user in the next period.

Preferably, the determining, based on the latest value and the predicted change value, an index target of the health index parameter of the user in the next period includes: determining, based on the latest value and the predicted change value, an estimate value of the health index parameter in the next period; determining whether the estimate value is greater than a control threshold upper limit corresponding to the health index parameter; and determining, based on the determining result, an index target of the health index parameter of the user in the next period.

Preferably, the determining, based on the determining result, an index target of the health index parameter of the user in the next period includes: if the determining result represents that the estimate value is greater than the control threshold upper limit, determining the estimate value as an upper limit value of the index target and using a lower limit of a corresponding normal range of the health index parameter as a lower limit value of the index target to obtain the index target of the health index parameter of the user in the next period; and if the determining result represents that the estimate value is not greater than the control threshold upper limit, using a normal range of the health index parameter as the index target of the health index parameter of the user in the next period.

Preferably, the method further includes: determining, based on the basic physical data of the user, a baseline value and a control threshold upper limit of the health index parameter; determining whether the baseline value is greater than the control threshold upper limit; and determining, based on the determining result, an initial target of the health index parameter of the user in an initial period.

Preferably, the determining, based on the determining result, an initial target of the health index parameter of the user in an initial period includes: if the baseline value is greater than the control threshold upper limit, using the control threshold upper limit as an upper limit value of the initial target and using a lower limit of a corresponding normal range of the health index parameter as a lower limit value of the initial target to obtain the initial target; and if the baseline value is not greater than the control threshold upper limit, using a normal range of the health index parameter as the initial target.

Preferably, the behavior guidance solution includes a dietary behavior guidance solution, and/or an exercise behavior guidance solution, and/or a drug administration behavior guidance solution.

According to a second aspect of the present application, a periodic behavior report generation apparatus is provided and includes: a first obtaining unit, configured to obtain behavior data and a health index parameter of a user in a predetermined period, the behavior data including at least one of an exercise behavior, a dietary behavior, or a drug administration behavior; a generating unit, configured to generate, according to the behavior data and the health index parameter, a periodic behavior report in the predetermined period for the user by at least partially using a machine learning model, the periodic behavior report including at least an evaluation result in the predetermined period, a behavior suggestion of a next period, and/or an index target of a next period; a second obtaining unit, configured to update, based on the periodic behavior report, a behavior label of the user, and obtain a patient education content matching with the behavior label; and a sending unit, configured to send the periodic behavior report and the patient education content to the user.

Preferably, the generating unit is further configured to: input the exercise behavior into a preset exercise behavior model, to obtain an evaluation result of the exercise behavior of the user, and generate an exercise behavior suggestion of the next period for the user based on the evaluation result; where the exercise behavior model includes a plurality of sub-models and an integration module, and the integration module is configured to determine the evaluation result according to outputs of the plurality of sub-models.

Preferably, the generating unit is further configured to: obtain a dietary content image of the user, input the dietary content image into a dietary behavior model, to obtain an evaluation result of the dietary behavior of the user, and generate a dietary behavior suggestion of the next period for the user based on the evaluation result, where the dietary behavior model is a convolutional neural network.

Preferably, the generating unit is further configured to: compare a blood glucose index and/or body composition index in the health index parameter with corresponding thresholds, to obtain an evaluation result of the blood glucose index and/or body composition; and invoke a corresponding behavior suggestion template for the user according to the evaluation result and by combining the behavior data, to generate an index behavior suggestion of the next period for the user.

Preferably, the second obtaining unit is further configured to: generate a personal label of the user based on the behavior data and the health index parameter of the user using an entity recognition algorithm; and perform label matching in a patient education content label library according to the personal label and use a patient education content of a highest degree of matching corresponding to at least one patient education content label as a matching patient education content.

Preferably, the generating unit is further configured to: obtain a latest value of the health index parameter generated by executing, by the user, a behavior guidance solution corresponding to the predetermined period in the predetermined period; obtain basic physical data of the user, a current value of the health index parameter, and an execution result of executing, by the user, the behavior guidance solution corresponding to the predetermined period in the predetermined period; perform prediction processing on the current value, the basic physical data, and the execution result using a model, to obtain a predicted change value of a health index parameter of the user in the next period; and determine, based on the latest value and the predicted change value, an index target of the health index parameter of the user in the next period.

Preferably, the generating unit is further configured to: determine, based on the latest value and the predicted change value, an estimate value of the health index parameter in the next period; determine whether the estimate value is greater than a control threshold upper limit corresponding to the health index parameter; and determine, based on the determining result, an index target of the health index parameter of the user in the next period.

According to a third aspect of the present application, a periodic behavior report generation system is provided and includes: a client, a server, and a database; where: the client obtains behavior data and a health index parameter of a user in a predetermined period, the behavior data including at least one of an exercise behavior, a dietary behavior, or a drug administration behavior; and the server obtains behavior data and a health index parameter from the client, generates, according to the behavior data and the health index parameter, a periodic behavior report in the predetermined period for the user by at least partially using a machine learning model, the periodic behavior report including at least an evaluation result in the predetermined period, a behavior suggestion of a next period, and/or an index target of a next period, updates, based on the periodic behavior report, a behavior label of the user, obtains a patient education content matching with the behavior label, and sends the periodic behavior report and the patient education content to the client.

According to a fourth aspect of the present application, an electronic device is provided and includes: a processor, a memory, and programs or instructions stored on the memory and capable of running on the processor, where when executed by the processor, the programs or instructions execute the steps of the periodic behavior report generation method.

According to a fifth aspect of the present application, a readable non-transient storage medium, storing programs or instructions, where when executed by a processor, the programs or instructions execute the steps of the periodic behavior report generation method.

For the periodic behavior report generation method, apparatus, and system, the storage medium, and the electronic device provided in the embodiments of the present application, by collecting the behavior data and health index parameter of the user, and inputting the behavior data and health index parameter of the user to the machine learning model for intelligent analysis, the periodic behavior report in the predetermined period is generated for the suer, and based on the periodic behavior report, the behavior label of the user is updated, to obtain the patient education content matching the behavior label, so as to help the user timely understand own guidance and suggestion in the process of health index parameter management, facilitating the user to guide own behavior based on the patient education content and the like. The embodiments of the present application achieve intelligent analysis of the behavior data and health index parameter of the user, and can determine bad behaviors in daily life of the user and give reasonable behavior suggestions based on physiological parameters of the user and specific behaviors of the user such as exercise, diet, and drug administration more scientifically. Therefore, different users can be accurately recommended for their own suitable behavior and dietary recommendations, which greatly improves the effect of behavior intervention and the control of physiological parameters of the user, and improves user experiences.

### BRIEF DESCRIPTION OF THE DRAWINGS

FIG. 1 is a flowchart of a periodic behavior report generation method provided by an embodiment of the present application.

FIG. 2 is an exemplary flowchart of a periodic behavior report generation method provided by an embodiment of the present application.

FIG. 3 is a flowchart of user behavior analysis provided by an embodiment of the present application.

FIG. 4 is a flowchart of user dietary analysis provided by an embodiment of the present application.

FIG. 5 is a schematic diagram of a composition structure of a periodic behavior report generation apparatus provided by an embodiment of the present application.

FIG. 6 is a schematic diagram of a structural graph of a periodic behavior report generation system provided by an embodiment of the present application.

FIG. 7 is a structural graph of an electronic device according to an embodiment of the present application.

### DETAILED DESCRIPTION

Combining with examples below, essence of technical solutions of the embodiments of the present application are elaborated in detail.

FIG. 1 is a flowchart of a periodic behavior report generation method provided by an embodiment of the present application. As shown in FIG. 1, the periodic behavior report generation method of the embodiment of the present application includes the following processing steps.

At step 101, behavior data and a health index parameter of a user in a predetermined period are obtained.

In the embodiment of the present application, the behavior data includes at least one of an exercise behavior, a dietary behavior, or a drug administration behavior.

The exercise behavior of the user includes outdoor or indoor exercise conditions of the user such as running, swimming, sleeping, and other conditions; these exercise behaviors can be obtained by inputting and uploading by the user him/herself, can also be obtained by collecting the exercise behaviors of the user through related applications such as through a pedometer, a heart rate monitor, or other manners, or is determined by performing exercise analysis on video captured images. The dietary behavior includes the type of food the user eats, the amount of food, the saltiness of the taste, etc. The drug administration behavior includes whether the user uses drugs, which drugs are used, drug dosages, frequency, and other information. The health index parameter of the user includes the user's age, gender, weight, height, heart rate, blood pressure, blood glucose, blood lipids, whether having a certain medical history, surgery, or serious disease history, and other data. Blood glucose index and weight are used as examples in the embodiment of the present application, which should not be understood as the limitation of the technical solution of the embodiment of the present application.

In the embodiment of the present application, the collected behavior data and health index parameter of the user need to be preprocessed, so that better prediction results can be obtained during the training of the data collected above. Preprocessing includes formatting normalization, scaling the image to a set pixel size for the image, etc. For the collected related data, obvious error values are deleted, etc.

Specifically, accuracy check is performed on the obtained data, and inaccurate data is modified, or the inaccurate user data is deleted. For example, the health index parameter is compared with an effective range corresponding to each index parameter, and whether the data is accurate is determined according to a comparison result. If the data is inaccurate, wrong data is further modified or deleted according to the comparison result.

For example, if a height of a certain adult user is 120 cm, and the height does not meet the requirements of the embodiment of the present application to the data, the user data is deleted. Alternately, if the blood pressure of a certain is 200, the blood pressure data does not fall within the effective range of blood pressure, and blood pressure data of the user is likely to be data with measurement problems, the user data is deleted.

For example, the common units of blood glucose are mg/dL and mmol/L, and the effective ranges of different blood glucose units are different. For example, the blood glucose data of a certain user is 120 mmol/L, which is obviously far beyond the effective blood glucose range, and the data is probably caused by the wrong unit. For example, if the blood glucose unit is modified to mg/dL or is converted according to a conversion relationship between the two units, the blood glucose data is modified to 120 mg/dL or 6.67 mmol/L. For another example, weight data of a certain male user is 140 (the unit is kg), but waist circumference data of the user is normal. At this time, it is possible that the unit of the weight data is “jin”, so the weight data is amended to 70 kg.

In a case that the data has a missing value, the data with the missing value is filled, for example, the missing data is filled with a mean value or a modal number. If enough data is obtained, the data with the missing value is deleted.

At step 102: a periodic behavior report in the predetermined period for the user is generated according to the behavior data and the health index parameter by at least partially using a machine learning model.

In the embodiment of the present application, the periodic behavior report includes at least an evaluation result in the predetermined period, a behavior suggestion of a next period, and/or an index target of a next period. For example, the periodic behavior report may include the evaluation result of the dietary behavior in the predetermined period and the dietary behavior suggestion of the next period; the evaluation result of the exercise behavior in the predetermined period and the exercise behavior suggestion of the next period; and the evaluation result of the health index parameter in the predetermined period, the index behavior suggestion of the next period, and the index target of the next period.

One period may be, for example, one day, one week, one month, or any other time length, which is not limited in the embodiment of the present application.

In the embodiment of the present application, the machine learning model may be a processing module of a pre-trained neural network and the like, which has functions of performing comprehensively intelligent analysis on change conditions of each behavior and/or health index parameter, for example, blood glucose based on the behavior data, dietary data, and drug administration data of the user above and by combining the health index parameter of the user and the like, determining for the user exercise, dietary, and drug administration behavior suggestions adapted to the user, and guiding the daily life behavior of the user based on the recommended behavior suggestions, thereby facilitating the user to living in a healthier manner and facilitating more control of the health index parameter of the user.

As an implementation the generating a periodic behavior report in the predetermined period for the user includes: inputting the exercise behavior into a preset exercise behavior model, to obtain an evaluation result of the exercise behavior of the user, and generating an exercise behavior suggestion of the next period for the user based on the evaluation result; where the exercise behavior model includes a plurality of sub-models and an integration module, and the integration module is configured to determine the evaluation result according to outputs of the plurality of sub-models.

As an example, the plurality of sub-models may include a logistic regression model, a Gradient Boosting Decision Tree (GBDT) model, a Random Forest (RF) model, a Shallow Neural Networks (SNN), etc., but are not limited thereto.

Each sub-model is separately trained using the data evaluation result as the target; after completing parameter tuning of each model above, the evaluation result is determined by the integrated model in a preferential manner. The logistic regression model obtains a dependent variable by inputting feature data, i.e., predicting the evaluation result. The GBDT model trains the GBDT train according to samples; for leaf nodes of each GBDT tree, a set of combination features can all be obtained by tracing back to a root node, and mark numbers of the leaf nodes are used as new combination features, so as to obtain the trained feature vector. For each training tree, the RF model uses corresponding out-of-bag data to calculate a classification error; features of all samples of the out-of-bag data are added with noises (randomly changing the values of the features), to further calculate the classification error. Hence, feature importance is determined. A shallow neural network can rapidly respond to the training data, obtain the feature vector, and classify the feature vector.

As an implementation, the integration module includes a bagging module. Specifically, a bagging mode is used for combining evaluation results of models together and outputting an evaluation result of the user exercise behavior.

In the embodiment of the present application, the evaluation result of the exercise behavior includes whether the exercise behavior reaches the standard, such as a relatively low exercise amount, a suitable exercise amount, and an excessive exercise amount. The exercise behavior suggestion corresponding to the relatively low exercise amount is to improve the strength; the exercise behavior suggestion corresponding to the suitable exercise amount is to maintain the strength; the exercise behavior suggestion corresponding to the excessive exercise amount is to lower the strength. In addition, the evaluation result of the embodiment of the present application further includes the evaluation on the drug administration behavior, such as an excessive drug dosage, a suitable drug dosage, and an insufficient drug dosage, or includes evaluation results such as the diet is too salty, too greasy, or excessive.

Alternatively, as an implementation, the generating a periodic behavior report in the predetermined period for the user includes: obtaining a dietary content image of the user, inputting the dietary content image into a dietary behavior model, to obtain an evaluation result of the dietary behavior of the user, and generating a dietary behavior suggestion for the user based on the evaluation result, where the dietary behavior model is a convolutional neural network.

Specifically, the dietary content image of the user is obtained; the dietary content image is scaled to the set the length-width size, the scaled image pixels are input into the neural network; through the training layer number set in the neural network, convolution and ReLU function processing are respectively performed on the input pixel features layer by layer, and then pooling processing is performed. The multi-path image pixel data upon pooling is subjected to Flatten processing, and then subjected to DenseNet classification, to output the evaluation result of the dietary behavior of the user.

The dietary behavior evaluation result herein includes at least one of the following: whether the diet of the user is greasy, whether to be excessive, whether to be too salty, and other evaluation results. The evaluation results are not limited thereto, but are only for exemplary explanations. For the evaluation result of the dietary behavior of the user, a corresponding dietary behavior suggestion is generated for the user, for example, for the evaluation result that the diet is too greasy, a suggestion for light diet and reducing and controlling intake of greasy food in the next period is generated for the user. For the evaluation result of excessive diet of the user, a dietary behavior suggestion for reducing eating amount for each meal, reducing the intake of snacks, and controlling the times for eating in the next period is generated for the user. For the evaluation result of salty diet of the user, a dietary behavior suggestion for light diet and reducing addition of salt and condiments in the food in the next period for the user.

Alternatively, as an implementation, the generating a periodic behavior report in the predetermined period for the user includes: comparing the health index parameter (for example, a blood glucose index/body composition index) with corresponding thresholds, to obtain an evaluation result; and invoking a corresponding behavior suggestion template for the user according to the evaluation result and by combining the behavior data, as an index behavior suggestion for the user.

At step 103: a behavior label of the user is updated based on the periodic behavior report, and a patient education content matching with the behavior label is obtained.

In some embodiments, the predetermined period may be one day, i.e., monitoring and performing behavior recommendation for the daily behavior of the user, for example, using as daily brief summary information of the user, to help the user with high blood glucose to know the blood glucose change condition, body composition change condition, exercise execution condition, and dietary execution condition of the user in time during the blood glucose management process. The daily brief summary relates to analyzing the dietary and exercise execution conditions of the previous day according to a current Fasting Blood Glucose (FBG), the blood glucose 2 hours after taking the meal, weight, body fat rate, muscle mass, and other index data conditions of the user, and providing clear guidance and suggestions.

In the embodiment of the present application, the predetermined period may also be other time lengths, such as 4 hours, 8 hours, 2 days, 3 days, and 8 days.

At step 104: the periodic behavior report and the patient education content is sent to the user.

In the embodiment of the present application, the predetermined period may be one week, i.e., monitoring and performing behavior recommendation for the weekly behavior of the user, for example, using as daily brief summary information of the user, to help the user with high blood glucose to know the blood glucose change condition, body composition change condition, exercise execution condition, and dietary execution condition of the user in time during the blood glucose management process. The weekly brief summary relates to comprehensively analyzing the blood glucose, body composition, dietary record, and exercise record data of the user in one week and providing clear guidance and suggestions, blood glucose monitoring plan of the next week, and other information.

In the embodiment of the present application, the predetermined period may also be one month, 2 weeks, 3 weeks, and other time lengths. It is specifically adjusted according to monitoring requirements of the user.

In the embodiment of the present application, when the behavior suggestion is determined for the user, after the arrival of the period, the behavior suggestion determined for the user is output to the user, to facilitate the user to refer to the suggestion for exercise, diet, drug administration, etc.

An embodiment of the present application includes: generating a personal label of the user based on the behavior data and the health index parameter of the user using an entity recognition algorithm; and performing label matching in a patient education content label library according to the personal label and using a patient education content of a highest degree of matching corresponding to at least one patient education content label as a matching patient education content. The patient education content label in the patient education content label library is obtained in the following way: using an entity recognition algorithm to recognize each patient education content and generating a patient education content label.

In the embodiment of the present application, when receiving selection and use information of the patient education content recommended for the user, it means that the relative information recommended to the user is confirmed by the user; the embodiment of the present application further supports to send feedback information to the user for statistics; a weight value of the content corresponding to the feedback information is adjusted based on the feedback information of the user, i.e., a feedback result of the recommended patient education content of the user can be used for adjusting a recommendation weight of the patient education content, so as to obtain more accurate patient education content results for the user.

In the embodiment of the present application, as a supplement to the method above, according to the historical exercise behavior, historical dietary behavior, and historical drug administration behavior of the user, an average value and variance information of the first health index parameter are determined; in a case of determining that the detection index of the first health index parameter of the user exceeds the range of positive and negative twice of the variance of the average value, alarm prompt information is given to the user. That is to say, when the first health index parameter is the blood glucose value, for the user, it is relatively dangerous when the blood glucose is higher than or lower than the set value; in a case of detecting that the blood glucose value of the user exceeds the positive and negative twice of the variance of the average value, it is required to notify the user to adopt a corresponding behavior instantly, to avoid discomforting the body of the user or causing an irreparable bodily harm.

In the embodiment of the present application, at the initial period of user behavior collection and behavior evaluation, since the behavior data or health index parameter data of the user are relatively few, threshold upper and lower limits implemented based on benchmarking simple rules through these values are used as the behavior analysis and evaluation basis for the user. As data accumulation for 2 weeks or longer time, the embodiment of the present application may calculate the average value and variance information of the patient regarding this index for each patient, and may monitor and perform behavior guidance on the health index parameter of the user according to the long-term average value and variance value.

In the embodiment of the present application, generating the period behavior report in the predetermined period for the user further includes generating an index target of the health index parameter in the next period for the user.

Specifically, it includes obtaining a latest value of the health index parameter generated by executing, by the user, a behavior guidance solution corresponding to the predetermined period in the predetermined period; and obtaining basic physical data of the user, a current value of the health index parameter, and an execution result of executing, by the user, the corresponding behavior guidance solution in the predetermined period; the behavior guidance solution includes the dietary behavior guidance solution, and/or exercise behavior guidance solution, and/or drug administration behavior guidance solution. Then a model is used for performing prediction processing on the current value, the basic physical data, and the execution result, to obtain a predicted change value of the health index parameter of the user in the next period; where the model may be any neural network model, for example, a shallow neural network model.

The initial target of the health index parameter generated by the user when executing the behavior guidance solution in the last period is determined. The initial target may be obtained based on prediction and may also be obtained by using the model to process the basic data of the user, or may be obtained after analyzing and processing the basic data of the user, which is not excessively limited herein. The health index parameter may be determined according to actual application scenes of the index target generating method; the health index parameter may be, for example, FBG, PBG, weight, blood pressure, or the like, which is not excessively limited herein.

The behavior guidance solution may be a preset behavior guidance solution and may also be the behavior guidance solution determined based on the initial target and the user basic physical data. The behavior guidance solution may be changed along with the change of the target of the user health index parameter, for example, the behavior guidance solution in the last period is for the last period and the behavior guidance solution in the next period corresponding to the index target in the next period. The dietary and exercise solutions of each period may be arranged by a doctor and may also calculated and adjusted by an AI.

The basic physical data includes, but not limited to, age, gender, height, weight, body fat, waistline, pancreas islet function, blood glucose, blood pressure, and the like. The current value of the health index parameter can be the latest value of the health index parameter at the end of the last period or the latest value of the health index parameter at the beginning of the next period.

Herein, analysis and processing can be performed on the basic physical data, the current value of health index parameter, and the execution result of the behavior guidance solution of the user in the last period to obtain the predicted change value of the health index parameter of the user in the next period. The machine learning method can also be used for predicting the predicted change value of the health index parameter in the next period.

Specifically, in a training stage, the basic physical data of the user, the current value of health index parameter, and the execution result of the behavior guidance solution of the user in the last period are taken together as training samples, and the predicted change value of the health index parameter at the end of the last period is taken as the training target. The shallow neural network is used for performing model training on the training samples to obtain training results. Based on a plurality of training results, the prediction model is obtained by adjusting the parameters of the model. In a prediction stage: the prediction model is used for preprocessing the sample to be tested of the user in the last period, to obtain the predicted change value of the health index parameter of the user in the next period. The sample to be tested in the last period includes basic physical data, a current value of the health index parameter, and an execution result of the behavior guidance solution of the user in the last period. For example, when the health index parameter is height, a height predicted change value is obtained after using the prediction model for processing. When the health index parameter is FBG, an FBG predicted change value is obtained after using the prediction model for processing. When the health index parameter is Postprandial Blood Glucose (PBG), a PBG predicted change value is obtained after using the prediction model for processing.

It should be explained that a standard behavior guidance solution can also be added into the training samples in the training stage and the samples to be tested in the prediction stage. The standard behavior guidance solution is different from the behavior guidance solution in the last period. The standard behavior guidance solution is the behavior guidance solution that the user's body is in an ideal state. The behavior guidance solution of the last period is a behavior guidance solution that comprehensively considers the user's physical condition and adapts to the initial target. For example, when the health index parameter is FBG or PBG, the standard behavior guidance solution is a blood glucose standard range, a weight standard range, a body fat standard range, and a muscle mass standard range. When the health index parameter is weight, the standard behavior guidance solution includes a standard dietary solution and a standard exercise solution. The standard dietary solution is that, for example, the nutrition ratio of the diet is balanced and the user can strictly follow the dietary solution; the standard exercise solution is that, for example, the exercise heart rate and exercise duration are both selected from the heart rate lower limit and the duration lower limit under the premise of meeting exercise requirements; usually, it is required that the user keeps the heart rate within a certain heart rate range and exercises for at least a certain duration requirement.

It should also be explained that the predicted change value is configured to indicate the increase or decrease amount in the health index parameter from the beginning of one period to the end of that period. In addition, the user of the present invention can be an ordinary user and can also be a patient with chronic diseases, such as the patient with type 2 diabetes or hypertension.

In this case, the next period is the period next to the last period; the last cycle can be the initial period and can also be any period for the user to execute the dietary and exercise solutions.

The embodiment of the present invention can dynamically adjust the index target of the next period according to a real execution result of the user in the last period, basic physical data, and the current value of the health index parameter, so as to enable the user, throughout the blood glucose lowering process, to make the target corresponding to each health index parameter more comply with the real physical condition of the user, so as to be more easily executed and achieved, thereby implementing the effective management of the target by the user and enhancing a sense of goal in blood glucose management by the user.

As an implementation, the determining, based on the latest value and the predicted change value, an index target of the health index parameter of the user in the next period includes: determining, based on the latest value and the predicted change value, an estimate value of the health index parameter in the next period; determining whether the estimate value is greater than a control threshold upper limit corresponding to the health index parameter; and determining, based on the determining result, an index target of the health index parameter of the user in the next period.

Specifically, if the determining result represents that the estimate value is greater than the control threshold upper limit, the estimate value is determined as an upper limit value of the index target and a lower limit of a corresponding normal range of the health index parameter is used as a lower limit value of the index target to obtain the index target of the health index parameter of the user in the next period; and if the determining result represents that the estimate value is not greater than the control threshold upper limit, a normal range of the health index parameter is used as the index target of the health index parameter of the user in the next period. For the health index parameter to correspond to the control threshold upper limit is determined according to the user basic physical data.

For example, when the generation method of this embodiment should be configured as: during the blood glucose control for a diabetic patient and the health index parameter is FBG, determining an upper limit of the FBG control threshold of the user based on the user's basic physical data such as age, gender, height, weight, body fat, waistline, islet function, and blood sugar; obtaining a difference between the latest value of FBG and the FBG predicted change value, and using the difference to determine an estimated value of FBG of the user in the next period; determining whether the estimated value is greater than a corresponding control threshold upper limit corresponding to the FBG, and if the determining result represents that the estimated value is greater than the corresponding control threshold upper limit corresponding to the FBG, using a lower limit of the normal range corresponding to the FBG as the lower limit value of the index target and using the estimated value as the upper limit value of the index target, so as to obtain the index target of the FBG of the user in the next period. If the determining result represents that the estimate value is not greater than the control threshold upper limit, a normal range of the FBG as the index target of the FBG of the user in the next period. Similarly, the determining process of the FBG index target of the third period is similar to the determining process of the FBG index target of the next period, and the difference is the index target of the FBG of the third period is obtained based on the data of the next period.

This embodiment can dynamically adjust the index target of the health index parameter based on the latest value of the health index parameter during the real execution process of the user, so that the making of the index target more complies with the real physical condition of the user, which not only enhances the sense of goal of the user and facilitates the execution of the target by the user, but also facilitates the user to effectively manage the health index parameter.

As an implementation, the method for determining an initial target of the user at least includes the following operation flow: determining, based on the basic physical data of the user, a baseline value and a control threshold upper limit of the health index parameter; determining whether the baseline value is greater than the control threshold upper limit; and determining, based on the determining result, an initial target of the health index parameter of the user in an initial period.

Specifically, if the baseline value is greater than the control threshold upper limit, the control threshold upper limit is used as an upper limit value of the initial target and a lower limit of a corresponding normal range of the health index parameter is used as a lower limit value of the initial target to obtain the initial target; and if the baseline value is not greater than the control threshold upper limit, a normal range of the health index parameter is used as the initial target.

Taking the health index parameter as FBG as an example, the FBG baseline value refers to the blood glucose value before the user is about to execute the initial periodic behavior guidance solution. This embodiment can determine the initial target of the initial periodic health index parameter based on the user's physical condition, so that the user can have a clear target at the beginning of implementing the behavior guidance solution, enhancing the user's sense of goal and improving the user's experience.

The health index target iterative generation method would be described in detail below by combining with type 2 diabetes.

The health index parameter of a patient with type 2 diabetes includes FBG, PBG, and weight. Since the iterative generation methods of FBG index target and PBG health index target are similar, the generations of FBG index target and weight index target are used as examples for illustration.

The FBG health index target iterative generation method is stated as follows:
first, according to the basic physical data of the patient, determining the FBG baseline value and the FBG control threshold upper limit, where the basic physical data of the patient includes body composition data (such as gender, age, height, and weight) and body index data (such as blood glucose and blood pressure). Whether the baseline value of the FBG is greater than the control threshold upper limit of the FBG is determined. If the determining result representing that the baseline value of the FBG is greater than the control threshold upper limit of the FBG, the control threshold upper limit of the FBG is used as an upper limit value of the initial target and a lower limit of a corresponding normal range of the FBG is used as a lower limit value of the initial target to obtain the initial target of the FBG of the user in the last period. If the baseline value of the FBG is not greater than the control threshold upper limit of the FBG, a normal range of the FBG is used as the initial target of the FBG of the user in the last period. For example, in a 7 days cycle, a patient with type 2 diabetes is older than 65 years old, has diabetes for more than 10 years, has high risk of hypoglycemia, has FBG of 8.2 mmol, and weighs 60 kg. After analyzing the basic physical data of the patient, the FBG baseline value is obtained to be 8.2 mmol, and the upper limit of the FBG control threshold is 6.9 mmol. Since the baseline value of the FBG is greater than the control threshold upper limit of the FBG, the initial target of the FBG of the user in the first 7 days is 4-8.2 mmol/L.

Secondly, the latest value of FBG generated by executing the behavior guidance solution for the last period by the patient in the last period is obtained; the basic physical data of the patient, the current value of FBG, the standard behavior guidance solution or behavior guidance solution, and/or the execution result of the behavior guidance solution by the user in the last period are obtained; the standard behavior guidance solution includes the blood glucose standard range, the weight standard range, the body fat standard range, and the muscle mass standard range; the trained prediction model is used for predicting the basic physical data, the current value of FBG, the behavior guidance solution or the standard behavior guidance solution, and the execution result, to obtain the FBG predicted change value of the user in the next period. For example, the machine learning mode is used for predicting the FBG reduction amount of the next period. The basic physical data of the patient, the current value of FBG, the blood glucose standard range, the weight standard range, the body fat standard range, the muscle mass standard range, and the execution result of the behavior guidance solution of the user in the last period are taken together as training samples, and the FBG change difference (for example, one week includes 7 days, the FBG value in the last period is the highest blood glucose in the 7 days minus the lowest blood glucose in the 7 days) of the patient in the last period is taken as the training target. The shallow neural network is used for performing model training on the training samples to obtain training results. Based on a plurality of training results, the prediction model is obtained by adjusting the parameters of the model. The prediction stage: the basic physical data of the patient, the current value of FBG, the blood glucose standard range, the weight standard range, the body fat standard range, the muscle mass standard range, and the execution result of the behavior guidance solution of the user in the last period are input into the prediction model to obtain the FBG change value of the user in the next period. Hence, combining the basic physical data of the user, the living habits, and current FBG as inputs of the prediction model; the FBG predicted change value is predicted; and the accuracy of FBG prediction is improved.

At last, based on the latest value of FBG and the FBG predicted change value, the FBG estimate value of the FBG in the next period is determined; whether the FBG estimate value is greater than the upper limit of the FBG control threshold is determined; if the determining result represents that the FBG estimate value is greater than the FBG control threshold upper limit, the FBG estimate value is used as an upper limit value of the index target and a lower limit of a corresponding normal range of the FBG is used as a lower limit value of the index target to obtain the index target of the FBG of the user in the next period; and if the determining result represents that the FBG estimate value is not greater than the FBG control threshold upper limit, a normal range of the FBG is used as the index target of the FBG of the user in the next period. For example, the latest value of FBG is 7.3 mmol, the FBG predicted change value is 0.1 mmol, and the upper limit of the FBG control threshold is 6.9 mmol. The FBG estimated value is 7.2 mmol. Since the FBG estimated value is greater than the upper limit of the FBG control threshold, the FBG index target of the user in the next period is 4.4-7.2 mmol.

The weight health index target iterative generation method is stated as follows:
first, according to the basic physical data of the patient, determining the weight baseline value and the weight control threshold upper limit, where the basic physical data of the patient includes body composition data (such as gender, age, height, and weight) and body index data (such as blood glucose and blood pressure). Whether the weight baseline value is greater than the weight control threshold upper limit is determined. If the determining result representing that the baseline value of the weight is greater than the control threshold upper limit of the weight, the control threshold upper limit of the weight is used as an upper limit value of the initial target and a lower limit of a corresponding normal range of the weight is used as a lower limit value of the initial target to obtain the initial target of the weight of the user in the last period. If the baseline value of the weight is not greater than the control threshold upper limit of the FBG, a normal range of the weight is used as the initial target of the weight of the patient in the last period. For example, in a 7 days cycle, a patient with type 2 diabetes is older than 65 years old, has diabetes for more than 10 years, has high risk of hypoglycemia, has FBG of 8.2 mmol, and weighs 60 kg. After analyzing the basic physical data of the patient, the weight baseline is obtained to be 60 kg, and the upper limit of the weight control threshold is 56 kg. Since the weight baseline value is greater than the weight control threshold upper limit, the initial target of the weight of the user in the first 7 days is 44.7-60 kg.

Next, the machine learning mode is used for predicting the weight predicted change value of the next period. The latest value of weight generated by executing the dietary and exercise solutions for the last period by the patient in the last period is obtained; the basic physical data of the patient, the current value of weight, the dietary and exercise solutions or standard dietary and exercise solutions, and/or the execution result of the dietary and exercise solutions by the user in the last period are obtained; the trained prediction model is used for predicting the basic physical data, the current value of weight, the dietary and exercise solutions or the standard dietary and exercise solutions, and/or the execution result, to obtain the weight predicted change value of the user in the next period. For example, the machine learning mode is used for predicting the weight reduction amount of the patient with type 2 diabetes in the next period. The basic physical data of the patient, the current value of weight, the dietary and exercise solutions or standard dietary and exercise solutions, and/or the execution result of the dietary and exercise solutions of the patient in the last period are taken together as training samples, and the weight difference of the user within 7 days (the highest weight in 7 days minus the lowest weight in 7 days) is taken as the training target. The shallow neural network is used for performing model training on the training samples to obtain training results. Based on a plurality of training results, the prediction model is obtained by adjusting the parameters of the model. The prediction stage: the basic physical data of the patient, the current value of the weight, the dietary and exercise solutions, or standard dietary and exercise solutions, and/or the execution result of the dietary and exercise solutions of the user in the first 7 days are input into the prediction model to obtain the weight predicted change value of a second 7 days of the user. Hence, combining the basic physical data of the user, the living habits, and weight current value as a model to be input; the weight predicted change value is predicted; and the accuracy of weight prediction is improved.

At last, based on the latest weight value and the weight predicted change value, the weight estimate value in the second 7 days is determined; whether the weight estimate value is greater than the weight control threshold upper limit is determined; if the determining result represents that the weight estimate value is greater than the weight control threshold upper limit, the weight estimate value is determined as an upper limit value of the index target and a lower limit of a corresponding normal range of the weight is used as a lower limit value of the index target to obtain the index target of weight of the user in the second 7 days; and if the determining result represents that the weight estimate value is not greater than the weight control threshold upper limit, a normal range of the weight is used as the index target of the weight of the user in the second 7 days. For example, the latest value of weight is 58 kg, the weight predicted change value output by the prediction model is 1 kg, and the upper limit of the weight control threshold is 56 kg. The weight estimated value is 57 kg. Since the weight estimated value is greater than the upper limit of the weight control threshold, the weight index target of the user in the second 7 days is 44.7-57 kg.

After repeated implementation of the behavior guidance solution, the FBG index target is 4.4-6.0 mmol/L and the weight index target is 47.4-55 kg after reaching the target in an n-th period.

It should be explained that the current value of weight can be the latest value of weight for the first 7 days, and can also be the latest value of weight for the first day at the beginning of the second 7 days. The current value of FBG can be the latest value of FBG for the first 7 days, and can also be the latest value of FBG for the first day at the beginning of the second 7 days.

Therefore, adopting the health index target iterative generation method in this embodiment is beneficial to the management of blood glucose in the patient with type 2 diabetes, so that the patient with type 2 diabetes can effectively control blood glucose according to the physical condition.

The present application would be further described in more details with reference to specific examples below.

In the embodiment of the present application, taking a patient with type 2 diabetes as an example for illustration, the monitoring of other health index parameters may still be conducted with reference to the technical solutions provided in the embodiment of the present application, and therefore, cannot be understood as the limitation of the technical solution of the embodiment of the present application. In the embodiment of the present application, the user adopts the form of a behavior brief summary to help the patient with type 2 diabetes to know the blood glucose change condition, body composition change condition, exercise execution condition, and dietary execution condition of the user in time during the blood glucose management process. The brief summary includes a daily brief summary and a weekly brief summary; the daily brief summary relates to analyzing the dietary and exercise execution conditions of the previous day according to an FBG, the blood glucose 2 hours after taking the meal, weight, body fat rate, muscle mass, and other index data conditions of the user in the morning of the current day, and providing clear guidance and suggestions. The weekly brief summary relates to comprehensively analyzing the blood glucose, body composition, dietary record, and exercise record data of the user in one week and providing clear guidance and suggestions, and blood glucose monitoring plan of the next week.

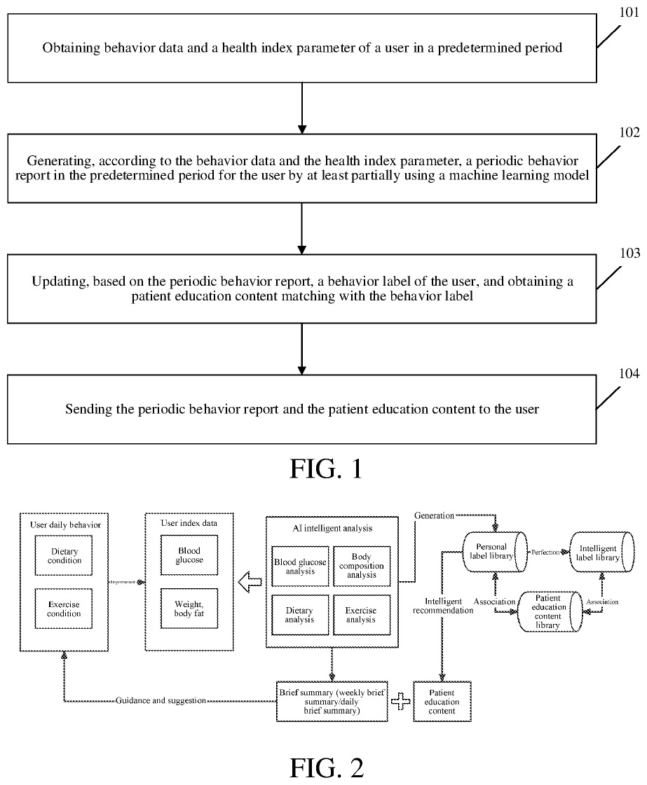

FIG. 2 is an exemplary flowchart of a periodic behavior report generation method provided by an embodiment of the present application. As shown in FIG. 2, the daily behaviors of the user are obtained, for example, including the dietary condition, exercise condition, and the like. The basic condition data of the user and the health index parameter, for example, the blood glucose, weight, body fat and other related parameters of the user, are obtained. The behavior data of the user, the basic condition date of the user, and the health index parameter are input into a preset machine learning model; a personal label library is analyzed and generated for the user through the machine learning model. In the embodiment of the present application, the personal label library mainly includes a correspondence between the daily behavior of the user and the corresponding blood glucose index, mainly including performing blood glucose analysis based on the collected blood glucose index, performing dietary condition analysis based on the dietary condition of the user, analyzing the obtained user body composition condition guidance information based on the body composition of the user, and performing exercise analysis based on the exercise condition of the user. In the embodiment of the present application, the intelligent analysis is performed on the user's behavior data and exercise data mainly based on a neural network technology, so as to determine the recommended behavior suitable for the user's own condition.

Specifically, a personal label of the user is generated based on the behavior data and the health index parameter of the user using an entity recognition algorithm. Personal label sets form a personal label library. Label matching is performed in the intelligent label library according to the personal labels. A patient education content corresponding to at least one intelligent label with the highest matching degree is used as the recommended content and the patient education content is output to the user. The intelligent label in the intelligent label library is obtained in the following way: using an entity recognition algorithm to recognize each patient education content and generating an intelligent label. Intelligent label sets constitute an intelligent label library.

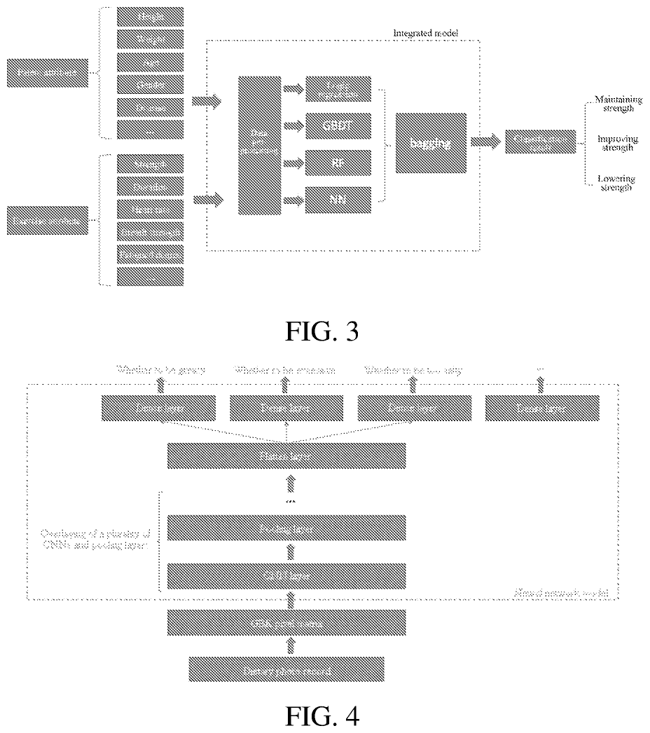

FIG. 3 is a flowchart of user behavior analysis provided by an embodiment of the present application. As shown in FIG. 3, the user behavior analysis process provided by the embodiment of the present application includes: after preprocessing the obtained behavior data and the basic condition data according to category, respectively inputting into a logistic regression model, a Gradient Promotion Decision Tree (GBDT) model, a Random Forests (RF) model, a Shallow Neural Network (SNN); separately training each model with a data evaluation result as the target; after finishing parameter tuning of each model, using bagging to combine the evaluation results of all models together to make behavior prediction for the user, which is used as the exercise behavior recommendation result of the user.

In the embodiment of the present application, the logistic regression, GBDT, RF, and SNN are four different prediction model algorithms; the models are respectively trained with an evaluation result as the target; after respectively finishing model tuning, a bagging idea is used for combining the model results together to make actual behavior prediction for the user, to facilitate the guidance for the user behavior.

FIG. 4 is a flowchart of user dietary analysis provided by an embodiment of the present application. As shown in FIG. 4, the user dietary analysis process of the embodiment of the present application includes: obtain a dietary content image of the user, after scaling the dietary content image to the set length and width, inputting pixels of the scaled image into the dietary behavior model. The dietary behavior model herein is a convolutional neural network. In the embodiment of the present application, through the training layer number set in the dietary behavior model, convolution and ReLU function processing are respectively performed on the input pixel features layer by layer. The multi-path image pixel data upon pooling is subjected to Flatten processing, and then subjected to DenseNet classification and evaluation, to generate the exercise behavior suggestion for the user in the next period based on the evaluation result.

In the embodiment of the present application, for the diet condition of the user, through the dietary behavior model diet photos uploaded by the user is classified and evaluated; the evaluation results of whether the diet of the user is greasy, whether to be excessive, whether to be salty, and the like are at least determined to perform knowledge recommendation subsequently based on the label generated by the evaluation result, facilitating to generate the dietary behavior suggestion and recommended patient education content for the user in the next period.

In the embodiment of the present application, for the diet image uploaded by the user, the user can be required to use a standard plate for taking the photo; during taking the photo, the plate area at least occupies a half of the image area, and the plate needs to be completely presented in the image, so as to determine whether the user's diet is excessive, and whether the carbohydrate therein is excessive, the amount of meat and vegetables in the image, and the like, so as to facilitate the analysis of the entire dietary condition of the user, so that the dietary information suitable for the user is determined by combining the user's blood glucose condition. In the embodiment of the present application, the saltiness of the diet can be voluntarily detected and reported by the user.

In the embodiment of the present application, for daily brief summary, at the initial period of user data processing, since the blood glucose or body composition data of the user are relatively few, threshold upper and lower limits implemented based on benchmarking simple rules through these values are used for managing the user behavior. As the data is accumulated (e.g., 2 weeks later), the average value and variance information of the user in this index can be calculated for each patient. In the future, when the new index of the user exceeds the range of “average value plus or minus 2 times of variance”, a warning prompt is output to the user. In the embodiment of the present application, depending on the characteristics of the index, setting a being-too-low warning if it is lower than “average value minus 2 times of variance”, or vice versa, i.e., setting a being-too-high warning can be selected.

When the user aims to reduce the blood glucose and lose weight, being-too-high warning is usually set. However, the possibility of being-too-low warning is not ruled out. For example, although blood glucose is decreasing and not below the lower limit of “low blood glucose”, if there is a medical possibility that “it should not decrease too fast”, it is considered that a warning is needed, otherwise no warning would be given.

As an example, exercise behavior suggestions can be generated for the user based on the exercise behavior model above, for example, generating that the current exercise intensity is appropriate, and is maintained and other suggested exercise items are continued; or outputting that the exercise intensity is too low, and the exercise intensity should be strengthened according to own physical condition; or outputting that the intensity is too high, the exercise intensity can be appropriately reduced. The specific exercise intensity can be reduced based on the user's exercise behavior. In the embodiment of the present application, the exercise recommendation mode above is used as the suggestion of the user's amount of exercise in the daily brief summary or weekly brief summary.

As an example, based on the dietary behavior training model, the dietary behavior suggestions can be output for the user, for example, the diet is relatively greasy, light diet should be taken tomorrow or next week; the diet amount is slightly excessive, and food intake should be controlled tomorrow or next week; the diet is relatively salty, and the diet salt intake should be reduced; and other evaluation results.

In addition, the blood glucose index in the user's health index parameter is compared with the standard blood glucose index to obtain the evaluation result. Based on the evaluation result and combining with the user's behavior data, suggestions on the user's exercise, diet, and the like are generated. That is, by collecting the user's blood glucose value, etc., the user's blood glucose control condition is directly determined, and corresponding suggestions for the next predetermined period such as days or weeks are generated, for example, suggestions that if blood glucose control is poor, drug dosage should be adjusted, exercise should be strengthened, and carbohydrate intake should be reduced; or according to the user's blood lipid index, whether the user's blood lipid exceeds or approaches the medical critical value is determined, and through the comparison of result, a suggestion that whether light diet is needed or strengthening the exercise is output to the user. In the embodiment of the present application, the format of the daily brief summary or weekly brief summary is not limited, as long as based on the user's behavior data and health index parameter and through the processing of relevant models, the corresponding recommended exercise behavior is generated for user.

In the embodiment of the present application, in the form of a brief summary and with the aid of intelligent analysis means such as a neural network, it helps the patient with type 2 diabetes to know the blood glucose change condition, body composition change condition, exercise execution condition, and dietary execution condition of the user in time during the blood glucose management process, and can recommend appropriate adapted behavior mode thereto, so that the user can better manage own health condition.

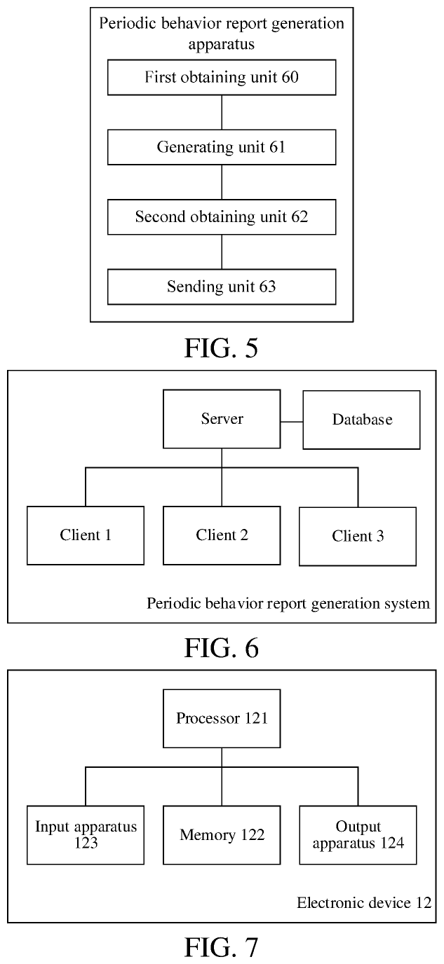

FIG. 5 is a schematic diagram of a composition structure of a periodic behavior report generation apparatus provided by an embodiment of the present application. As shown in FIG. 5, the periodic behavior report generation apparatus of the embodiment of the present application includes: a first obtaining unit 60, configured to obtain behavior data and a health index parameter of a user in a predetermined period, the behavior data including at least one of an exercise behavior, a dietary behavior, or a drug administration behavior; a generating unit 61, configured to generate, according to the behavior data and the health index parameter, a periodic behavior report in the predetermined period for the user by at least partially using a machine learning model, the periodic behavior report including at least an evaluation result in the predetermined period, a behavior suggestion of a next period, and/or an index target of a next period; a second obtaining unit 62, configured to update, based on the periodic behavior report, a behavior label of the user, and obtain a patient education content matching with the behavior label; and a sending unit 63, configured to send the periodic behavior report and the patient education content to the user.

In an embodiment of the present application, the generating unit 61 is further configured to: input the exercise behavior into a preset exercise behavior model, to obtain an evaluation result of the exercise behavior of the user, and generate an exercise behavior suggestion of the next period for the user based on the evaluation result; where the exercise behavior model includes a plurality of sub-models and an integration module, and the integration module is configured to determine the evaluation result according to outputs of the plurality of sub-models.

In an embodiment of the present application, the generating unit 61 is further configured to: obtain a dietary content image of the user, input the dietary content image into a dietary behavior model, to obtain an evaluation result of the dietary behavior of the user, and generate a dietary behavior suggestion of the next period for the user based on the evaluation result, where the dietary behavior model is a convolutional neural network.

In an embodiment of the present application, the generating unit 61 is further configured to: compare a blood glucose index and/or body composition index in the health index parameter with corresponding thresholds, to obtain an evaluation result of the blood glucose index and/or body composition; and invoke a corresponding behavior suggestion template for the user according to the evaluation result and by combining the behavior data, to generate an index behavior suggestion of the next period for the user.

In an embodiment of the present application, the second obtaining unit 62 is further configured to: generate a personal label of the user based on the behavior data and the health index parameter of the user using an entity recognition algorithm; and perform label matching in a patient education content label library according to the personal label and use a patient education content of a highest degree of matching corresponding to at least one patient education content label as a matching patient education content.

In an embodiment of the present application, the generating unit 61 is further configured to: obtain a latest value of the health index parameter generated by executing, by the user, a behavior guidance solution corresponding to the predetermined period in the predetermined period; obtain basic physical data of the user, a current value of the health index parameter, and an execution result of executing, by the user, the behavior guidance solution corresponding to the predetermined period in the predetermined period; perform prediction processing on the current value, the basic physical data, and the execution result using a model, to obtain a predicted change value of a health index parameter of the user in the next period; and determine, based on the latest value and the predicted change value, an index target of the health index parameter of the user in the next period.

In an embodiment of the present application, the generating unit 61 is further configured to: determine, based on the latest value and the predicted change value, an estimate value of the health index parameter in the next period; determine whether the estimate value is greater than a control threshold upper limit corresponding to the health index parameter; and determine, based on the determining result, an index target of the health index parameter of the user in the next period.

In an embodiment of the present application, the generating unit 61 is further configured to: if the determining result represents that the estimate value is greater than the control threshold upper limit, determine the estimate value as an upper limit value of the index target and use a lower limit of a corresponding normal range of the health index parameter as a lower limit value of the index target to obtain the index target of the health index parameter of the user in the next period; and if the determining result represents that the estimate value is not greater than the control threshold upper limit, use a normal range of the health index parameter as the index target of the health index parameter of the user in the next period.

In an embodiment of the present application, the generating unit 61 is further configured to: determine, based on the basic physical data of the user, a baseline value and a control threshold upper limit of the health index parameter; determine whether the baseline value is greater than the control threshold upper limit; and determine, based on the determining result, an initial target of the health index parameter of the user in an initial period.

In an embodiment of the present application, the generating unit 61 is further configured to: if the baseline value is greater than the control threshold upper limit, use the control threshold upper limit as an upper limit value of the initial target and use a lower limit of a corresponding normal range of the health index parameter as a lower limit value of the initial target to obtain the initial target; and if the baseline value is not greater than the control threshold upper limit, use a normal range of the health index parameter as the initial target.

In an embodiment of the present application, the generating unit 61 is further configured that: the behavior guidance solution includes a dietary solution and/or an exercise solution.

In an exemplary embodiment, the each processing unit of the periodic behavior report generation apparatus of the embodiment of the present application may be implemented by one or more of Central Processing Unit (CUP), Graphics Processing Unit (GPU), Base Processor (BP), Application Specific Integrated Circuit (ASIC), DSP, Programmable Logic Device (PLD), Complex Programmable Logic Device (CPLD), Field-Programmable Gate Array (FPGA), general-purpose processor, controller, Micro Controller Unit (MCU), microprocessor, or other electronic elements.

In the embodiment of the present disclosure, specific modes for each processing unit in the periodic behavior report generation apparatus shown in FIG. 5 to execute operations are described in detail in the embodiments relating to the method, which are not stated and explained in detail herein.

FIG. 6 is a schematic diagram of a structural graph of a periodic behavior report generation system provided by an embodiment of the present application. As shown in FIG. 6, the embodiment of the present application further recites a periodic behavior report generation system. The system includes: a client, a server, and a database; where the client obtains behavior data and a health index parameter of a user in a predetermined period, the behavior data including at least one of an exercise behavior, a dietary behavior, or a drug administration behavior; and the server obtains behavior data and a health index parameter from the client, generates, according to the behavior data and the health index parameter, a periodic behavior report in the predetermined period for the user by at least partially using a machine learning model, the periodic behavior report including at least an evaluation result in the predetermined period, a behavior suggestion of a next period, and/or an index target of a next period, updates, based on the periodic behavior report, a behavior label of the user, obtains a patient education content matching with the behavior label, and sends the periodic behavior report and the patient education content to the client. The client includes client 1, client 2, and client 3.

In the embodiment of the present application, the client and server are connected through a wired or wireless network; the client can be installed on the user's electronic devices such as mobile phones and laptops; the server side is provided with a patient education content library, user behavior label library, intelligent label library, and other databases to facilitate the server to call to the database. As a way of implementation, the periodic behavior report generation method of the embodiment of the present application is implemented by a software program, and related application programs can be installed on the electronic device of the user, i.e., directly implementing data collection, behavior recommendation, and the like by the electronic device at the user side without a network processing mode; the embodiment of the present application is not limited to the specific means of implementation.

The periodic behavior report generation system of the embodiment of the present application is described carried in detail in the preceding embodiment; unnecessary specific implementation details are not repeated herein; a person skilled in the art should understand that, based on the existing connection means and related technology, realizing the periodic behavior report generation system would be easily implemented. The system structure of the specific example of the embodiment of the present application shall not be taken as the limitation of the technical solution of the embodiment of the present application.

The electronic device 12 of the embodiment of the present application is described below with reference to FIG. 7 below.

As shown in FIG. 7, the electronic device 12 includes one or more processors 121 and a memory 122.

The processor 121 may be a central processing unit (CPU) or processing units in other forms with data processing capability and/or instruction execution capability, and may control other assemblies in the electronic device 12 to perform the desired functions.

The memory 122 may include one or more computer program products; the computer program products may include computer-readable storage media in various forms, such as volatile memory and/or non-volatile memory. The volatile memory may include, for example, a random access memory (RAM) and/or a cache. The non-volatile memory, for example, may include read-only memory (ROM), hard disk, flash memory, etc. One or more computer program instructions may be stored on the computer-readable storage medium, and processor 121 may run the program instructions to achieve the periodic behavior report generation method of each embodiment of the present application and/or other desired functions described above. The computer-readable storage medium can also store various contents such as input signal, signal component, and noise component.

In one example, the electronic device 12 could also include: an input apparatus 123 and an output apparatus 124. These assemblies are interconnected through a bus system and/or connection mechanisms in other forms (not shown in FIG. 7).

The input apparatus 123 may include, for example, a keyboard, mouse, etc.

The output apparatus 124 can output all kinds of information to the outside, including the determined distance information, direction information, etc. The output apparatus 124 may include, for example, displays, loudspeakers, printers, and communication networks, remote output devices connected thereto, etc.

Certainly, for simplicity, only some assemblies of the electronic device 12 relevant to the present application are shown in FIG. 7, omitting assemblies such as buses and input/output interfaces. In addition, the electronic device 12 may include any other appropriate assemblies, depending on specific application conditions.

The embodiment of the present application further recites a readable non-transient storage medium, storing programs or instructions, where when executed by a processor, the programs or instructions execute steps of the periodic behavior report generation method.

It should be understood that “one embodiment” or “an embodiment” mentioned throughout the specification means that a specific feature, structure, or characteristic related to the embodiment is included in at least one embodiment of the present application. Hence, “in one embodiment” or “in an embodiment” appearing throughout the entire specification does not necessarily refer to the same embodiment. In addition, the specific features, structures, or characteristics can be combined into one or more embodiments in any proper mode. It should be understood that in the various embodiments of the present application, the size of the serial numbers of the processes above does not mean the order of execution; the order of execution of each process should be determined by its function and internal logic and should not constitute any limitation on the implementation process of the embodiment of the present application. The sequence numbers of the embodiments of this application are merely for description but do not imply the preference among the embodiments.

It should be explained that in this text, the terms “including”, “comprising” or any other variation thereof are intended to cover non-exclusive inclusion so that a process, method, article or apparatus that includes a set of elements includes not only those elements but also other elements that are not clearly listed, or further includes elements inherent to the process, method, article or apparatus. Without further restrictions, the elements defined by the sentence “including one . . . ” does not exclude the existence of other identical elements in the process, method, article or apparatus that includes the element.

In the several embodiments provided in the present application, it should be understood that, the disclosed device and method may be implemented in other manners. The device embodiments described above are merely exemplary. For example, the division of the units is merely the division of logic functions, and may use other division manners during actual implementation. For example, a plurality of units or assemblies may be combined, or may be integrated into another system, or some features may be omitted or not performed. In addition, coupling, or direct coupling, or communicative connection between the displayed or discussed components may be indirect coupling or communicative connection through some interfaces, devices, or units, and may be electrical, mechanical, or of other forms.

The parts described as separate parts may or may not be physically separated, and parts displayed as units may or may not be physical units, and may be located in one place or may be distributed over a plurality of network units. Some or all of the units can be selected according to the actual needs to achieve the purpose of this embodiment solution.

In addition, functional units in the embodiments of the present application may be integrated into one processing unit, or each of the units may be separately used as one unit, or two or more units may be integrated into one unit. The integrated units can be implemented both in the form of hardware as well as hardware and software functional units.

The disclosed are only implementations of the present application; however, the scope of protection of the present application is not limited herein. Any person skilled in the technical field could easily conceive of variations and replacements in the technical scopes of the present application, and they should all be covered in the scope of protection of the present application. Therefore, the scope of protection of the present application shall be subject to the appended claims.

1-15. (canceled)

16. A periodic behavior report generation method, comprising:
obtaining behavior data and a health index parameter of a user in a predetermined period, the behavior data comprising at least one of an exercise behavior, a dietary behavior, or a drug administration behavior;
generating, according to the behavior data and the health index parameter, a periodic behavior report in the predetermined period for the user by at least partially using a machine learning model, the periodic behavior report comprising at least an evaluation result in the predetermined period, a behavior suggestion of a next period, and/or an index target of a next period;
updating, based on the periodic behavior report, a behavior label of the user, and obtaining a patient education content matching with the behavior label; and
sending the periodic behavior report and the patient education content to the user.

17. The method according to claim 16, wherein the generating a periodic behavior report in the predetermined period for the user comprises:
inputting the exercise behavior into a preset exercise behavior model, to obtain an evaluation result of the exercise behavior of the user, and generating an exercise behavior suggestion of the next period for the user based on the evaluation result;
wherein the exercise behavior model comprises a plurality of sub-models and an integration module, and the integration module is configured to determine the evaluation result according to outputs of the plurality of sub-models.

18. The method according to claim 16, wherein the generating a periodic behavior report in the predetermined period for the user comprises:
obtaining a dietary content image of the user, inputting the dietary content image into a dietary behavior model, to obtain an evaluation result of the dietary behavior of the user, and generating a dietary behavior suggestion of the next period for the user based on the evaluation result, wherein the dietary behavior model is a convolutional neural network.

19. The method according to claim 16, wherein the generating a periodic behavior report in the predetermined period for the user comprises:
comparing a blood glucose index and/or body composition index in the health index parameter with corresponding thresholds, to obtain an evaluation result of the blood glucose index and/or body composition; and invoking a corresponding behavior suggestion template for the user according to the evaluation result and by combining the behavior data, to generate an index behavior suggestion of the next period for the user.

20. The method according to claim 17, wherein the obtaining a patient education content matching with the behavior label comprises:
generating a personal label of the user based on the behavior data and the health index parameter of the user using an entity recognition algorithm; and
performing label matching in a patient education content label library according to the personal label and using a patient education content of a highest degree of matching corresponding to at least one patient education content label as a matching patient education content.

21. The method according to claim 16, wherein the generating a periodic behavior report in the predetermined period for the user comprises:
obtaining a latest value of the health index parameter generated by executing, by the user, a behavior guidance solution corresponding to the predetermined period in the predetermined period;
obtaining basic physical data of the user, a current value of the health index parameter, and an execution result of executing, by the user, the behavior guidance solution corresponding to the predetermined period in the predetermined period;
performing prediction processing on the current value, the basic physical data, and the execution result using a model, to obtain a predicted change value of a health index parameter of the user in the next period; and
determining, based on the latest value and the predicted change value, an index target of the health index parameter of the user in the next period.

22. The method according to claim 21, wherein the determining, based on the latest value and the predicted change value, an index target of the health index parameter of the user in the next period comprises:
determining, based on the latest value and the predicted change value, an estimate value of the health index parameter in the next period;
determining whether the estimate value is greater than a control threshold upper limit corresponding to the health index parameter; and
determining, based on the determining result, an index target of the health index parameter of the user in the next period.

23. The method according to claim 22, wherein the determining, based on the determining result, an index target of the health index parameter of the user in the next period comprises:
if the determining result represents that the estimate value is greater than the control threshold upper limit, determining the estimate value as an upper limit value of the index target and using a lower limit of a corresponding normal range of the health index parameter as a lower limit value of the index target to obtain the index target of the health index parameter of the user in the next period; and
if the determining result represents that the estimate value is not greater than the control threshold upper limit, using a normal range of the health index parameter as the index target of the health index parameter of the user in the next period.

24. The method according to claim 21, further comprising:
determining, based on the basic physical data of the user, a baseline value and a control threshold upper limit of the health index parameter;
determining whether the baseline value is greater than the control threshold upper limit; and
determining, based on the determining result, an initial target of the health index parameter of the user in an initial period.

25. The method according to claim 24, wherein the determining, based on the determining result, an initial target of the health index parameter of the user in an initial period comprises:
if the baseline value is greater than the control threshold upper limit, using the control threshold upper limit as an upper limit value of the initial target and using a lower limit of a corresponding normal range of the health index parameter as a lower limit value of the initial target to obtain the initial target; and
if the baseline value is not greater than the control threshold upper limit, using a normal range of the health index parameter as the initial target.

26. The method according to claim 21, wherein the behavior guidance solution comprises a dietary behavior guidance solution, and/or an exercise behavior guidance solution, and/or a drug administration behavior guidance solution.

27. A periodic behavior report generation apparatus, comprising:
a first obtaining unit, configured to obtain behavior data and a health index parameter of a user in a predetermined period, the behavior data comprising at least one of an exercise behavior, a dietary behavior, or a drug administration behavior;
a generating unit, configured to generate, according to the behavior data and the health index parameter, a periodic behavior report in the predetermined period for the user by at least partially using a machine learning model, the periodic behavior report comprising at least an evaluation result in the predetermined period, a behavior suggestion of a next period, and/or an index target of a next period;
a second obtaining unit, configured to update, based on the periodic behavior report, a behavior label of the user, and obtain a patient education content matching with the behavior label; and
a sending unit, configured to send the periodic behavior report and the patient education content to the user.

28. A periodic behavior report generation system, comprising a client, a server, and a database, wherein:
the client obtains behavior data and a health index parameter of a user in a predetermined period, the behavior data comprising at least one of an exercise behavior, a dietary behavior, or a drug administration behavior; and
the server obtains the behavior data and the health index parameter from the client, generates, according to the behavior data and the health index parameter, a periodic behavior report in the predetermined period for the user by at least partially using a machine learning model, the periodic behavior report comprising at least an evaluation result in the predetermined period, a behavior suggestion of a next period, and/or an index target of a next period, updates, based on the periodic behavior report, a behavior label of the user, obtains a patient education content matching with the behavior label, and sends the periodic behavior report and the patient education content to the client.

29. An electronic device, comprising a processor, a memory, and programs or instructions stored on the memory and capable of running on the processor, wherein when executed by the processor, the programs or instructions execute the steps of the periodic behavior report generation method according to claim 16.
